(19) United States
(12) Patent Application Publication
(10) Pub. No.: US 2024/0041714 A1
(43) Pub. Date: Feb. 8, 2024

(54) Oil-Based Cosmetic

(71) Applicant: JO COSMETICS CO., LTD., Tokyo (JP)

(72) Inventors: Takuma Inaka, Tokyo (JP); Yusuke Akizuki, Tokyo (JP); Ryuta Yuasa, Tokyo (JP)

(73) Assignee: JO COSMETICS CO., LTD., Tokyo (JP)

(21) Appl. No.: 18/268,998

(22) PCT Filed: Dec. 13, 2021
(86) PCT No.: PCT/JP2021/045733

§ 371 Date: Jun. 22, 2023

(30) Foreign Application Priority Data
Dec. 26, 2020 (JP) 2020-218078

(51) Int. Cl. A61K 8/06; A61K 8/893; A61K 8/63; A61K 8/60; A61K 8/25; A61Q 1/06; A61K 8/92; A61K 8/37

(52) U.S. Cl. CPC A61K 8/066; A61K 8/893; A61K 8/63; A61K 8/602; A61K 8/25; A61Q 1/06; A61K 8/922; A61K 8/37

(57) ABSTRACT

An oil-based cosmetic contains, as an oily base, an oil containing a phytosterol derivative, having a refractive index at 40° C. of 1.50 or more, and being liquid at 25° C. (A) and a liquid silicone oil that is incompatible with the oil (A) at 25° C. (B). In addition to the oily base material, a separation inhibitor to suppress separation of the oil of the component (A) and the silicone oil of the component (B) (C) is preferably contained, and a powder (D) and a solid oil and/or lipophilic gelling agent (E) may also be contained. A form of the of oil-based cosmetic can be appropriately selected according to an application, and can be a liquid, paste, solid, or the like.

### TECHNOLOGICAL FIELD

The present invention relates to an oil-based cosmetic having excellent color transfer resistance, color retention, gloss, and gloss persistence.

### BACKGROUND TECHNOLOGY

Conventionally, as for an oil-based cosmetic such as a lipstick, a color transfer property that the cosmetic is transferred to a contacted site when the cosmetic touches a contacting object such as clothes or a cup after being applied to the skin or lips (also referred to as “secondary adhesion”) has been a problem. Hence, it has been promoted to develop a cosmetic for lips that has less secondary adhesion, that is, a cosmetic for lips having an excellent color transfer resistance. For example, Patent Document 1 proposes a cosmetic for lips containing (a) hydrogenated polyisobutene and (b) methylphenylsilicone that separates when mixed with the component (a). This literature describes that the component (b) forms a continuous phase while the component (a) containing a colorant forms a dispersed phase, and that the component (b), which forms a continuous phase, separates to a surface layer upon application, thereby imparting effects of gloss and color transfer resistance (less secondary adhesion) (see) [0011]). This cosmetic has good gloss immediately after application since, when this is applied, methylphenylsilicone containing no colorant separates and forms a surface layer that is a smooth transparent layer. However, this cosmetic had a problem that after the surface layer is lost due to contact with an object such as clothes or a cup, the hydrogenated polyisobutene layer with a low refractive index is exposed, resulting in a decrease in gloss and gloss persistence.

On the other hand, Patent Document 2 describes an oil composition containing, as a main component, a steradiene that has a structure converted to 3,5-conjugated diene by dehydrating the hydroxyl group at 3-C of phytosterol and having a phytosterol content of 20% by mass or less; and an oily base for an external preparation for skin using the oil composition. This literature also describes that, when a steradiene content is high while a phytosterol content is low, the oil composition has a high refractive index, and that the oil composition exhibits an excellent gloss imparting property when used in an external preparation for skin (see paragraph 0008) and can be used for a lipstick and a lip gloss (see claim 10). This literature also describes that the oil composition may be used in combination with other oils such as silicone oils (see paragraph 0034). However, there is no disclosure of an embodiment in which the oil composition is used in combination with silicone oils, nor is there any disclosure relating to a combination of the oil composition and another oil incompatible therewith.

### PRIOR ART DOCUMENT

### Patent Document

Patent Document 1: Japanese Patent Laid-Open No. 2011-140481
Patent Document 2: Japanese Patent Laid-Open No. 2018-131404

### DISCLOSURE OF THE INVENTION

### Problem to be Solved by the Invention

The present invention was completed based on such a background art, and an object thereof is to provide an oil-based cosmetic excellent in color transfer resistance, color retention, gloss, and gloss persistence.

### Means Used to Solve the Problem

As a result of intensive studies to solve the above problem, the present inventors have found that, by using as an oily base an oil containing a phytosterol derivative having a refractive index at 40° C. of 1.50 or more and being a liquid at 25° C. in combination with a liquid silicone oil incompatible therewith, an oil-based cosmetic that is excellent in color transfer resistance, color retention, gloss, and gloss persistence can be obtained, and thus the present invention was completed.

Thus, according to the present invention, there is provided an oil-based cosmetic containing an oil containing a phytosterol derivative which has a refractive index at 40° C. of 1.50 or more and is liquid at 25° C. (A) and a liquid silicone oil which is not compatible with the oil (A) at 25° C. (B) as an oily base.

### Effect of the Invention

The oil-based cosmetic of the present invention is excellent in gloss and gloss persistence when applied to the skin or lips in addition to color transfer resistance and color retention.

### EMBODIMENT FOR CARRYING OUT THE INVENTION

The oil-based cosmetic of the present invention contains, as essential components, an oil that contains a phytosterol derivative, has a refractive index at 40° C. of 1.50 or more and is liquid at 25° C. (A) and a liquid silicone oil that is incompatible with the oil (A) at 25° C. (B).

The oil-based cosmetic of the present invention may be liquid or solid. It may have a single layer in which the oil of the component (A) and the silicone oil of the component (B) make a uniform mixture macroscopically (i.e., single-layer type), or it may have a plurality of layers composed of both components (multi-layer type). Examples of such a multi-layer type include a two-layer type consisting of a layer mainly composed of the oil of the component (A) and a layer mainly composed of the silicone oil of the component (B), a two-layer type consisting of a layer mainly composed of the oil of the component (A) and a layer in which the oil of the component (A) and the silicone oil of the component (B) make a uniform mixture macroscopically, and the like. In the case of the multi-layer type, it is necessary to mix and homogenize a plurality of layers prior to use. In view of such an aspect, the oil-based cosmetic is preferably the single-layer type. In the case of the single-layer cosmetic, the cosmetic is preferably in a form of an oil-in-oil emulsion. Particularly, an oil-in-oil emulsion in which the oil of the component (A) forms a continuous phase, and the silicone oil of the component (B) forms a dispersed phase is preferred. Since the oil of the component (A) is excellent in affinity with solid oils such as hydrocarbons and waxes, formation of a continuous layer by the oil of the component (A) imparts good shape retention (retention of shape such as a stick) when producing a solid cosmetic containing a solid oil.

The oil of the component (A) usually has a greater surface tension than the silicone oil of the component (B). Further, the oil of the component (A) and the silicone oil of the component (B) are incompatible. Therefore, it is believed that, upon applying the oil-based cosmetic of the present invention containing both components to the skin or lips, the silicone oil of the component (B) having a smaller surface tension separates and forms a surface layer, while the oil of the component (A) having a larger surface tension adheres to the skin side. When powders such as coloring powders and ultraviolet scattering agents are included, since the powders are mainly dispersed in the oil of the component (A) having good affinity for them, the silicone oil forms a surface layer that is smooth and almost transparent. As a result, the oil-based cosmetic is believed to be excellent in gloss. And since the silicone oil layer contains little coloring material causing color transfer (secondary adhesion), it is possible to prevent color transfer of the cosmetic even when touching an object such as clothes or a cup. Also, even after the silicone oil layer on the surface is lost due to contact with the clothes or cup, the oil-based cosmetic of the present invention can maintain good gloss since the residual oil has a refractive index higher than hydrogenated polyisobutene used in conventional oil-in-oil cosmetics, for example, the oil-in-oil cosmetic described in Patent Document 1.

The oil of the component (A) used in the present invention is an oil that contains a phytosterol derivative, has a refractive index at 40° C. of 1.50 or more, and is liquid at 25° C. Phytosterols, also referred to as plant sterols, are components that are contained in trace amounts in vegetable oils such as soybean oil and rapeseed oil and are a group of compounds that are classified as sterols (steroid alcohols). Although phytosterols are generally white solids with unique odors, derivatives thereof also include a compound being liquid at 25° C. such as steradiene described in Patent Document 2. It is noted that steradiene refers to a dehydrated phytosterol having a structure converted to 3,5-diene structure by dehydrating the hydroxyl group at 3-C of phytosterol. Such a steradiene can be obtained by subjecting a phytosterol to a dehydration reaction to dehydrate the hydroxyl group at 3-position, and a specific manufacturing method thereof is described in Patent Document 2.

The oil of the component (A) may be any oil as far as it contains a phytosterol derivative, has a refractive index at 40° C. of 1.50 or more, and is liquid at 25° C. When a content of the phytosterol derivative is low, a refractive index of the oil decreases. Therefore, it becomes difficult to make a cosmetic having a good gloss upon applying the cosmetic. Also, when the silicone layer is lost due to the secondary attachment, it becomes difficult to maintain the gloss. The oil of the component (A) is preferably an oily material containing steradiene as a main component. Since steradiene is usually obtained by dehydrating the hydroxyl group at 3-C of phytosterol, it sometimes contains an unreacted portion of phytosterol used as a starting material, other compounds contained as impurities in the starting material, and by-products in the dehydration reaction of phytosterol. These compounds may be included in a range where the effects of the present invention are not substantially impaired.

A refractive index at 40° C. of the oil of the component (A) is preferably 1.51 or more, more preferably 1.52 or more. When the refractive index is low, gloss is reduced when the cosmetic is applied, and gloss cannot be maintained when the surface layer composed of the silicone oil is lost due to the secondary attachment. The refractive index can be measured according to the refractive index measurement method, which is the general test method defined in Japanese Standards of quasi-drug ingredients 2006, using an Abbe refractometer (Model:NAR-2T, manufactured by ATAGO Co., Ltd). A viscosity at 25° C. of the oil of the component (A) is usually 20,000 to 60,000 mPa·s preferably 35,000 to 50,000 mPa·s. The viscosity can be measured using a BM-type viscometer that is manufactured by Toki Sangyo Co., Ltd. according to the viscosity measurement method (second method), which is the general test method defined in Japanese Standards of quasi-drug ingredients 2006. Examples of commercial products of the oil of the component (A) include TECHNOL SD having a refractive index of 1.52 (INCI name:Phytosterols) that is manufactured by Yokozeki Oil & Fat Industries Co. Ltd.

A content of the oil of the component (A) in the oil-based cosmetic of the present invention is preferably 5 to 70% by mass, more preferably 5 to 60% by mass, furthermore preferably 10 to 50% by mass. If the content of the oil of the component (A) is too small, gloss persistence is reduced, and if too large, spreadability upon application is reduced.

In the present invention, the oil of the component (A) and a liquid silicone oil which is incompatible therewith at 25° C. (B) are used as a component of an oily base. The liquid silicone oil used is not particularly limited as far as it is incompatible with the oil of the component (A) at 25° C. In this specification, whether “incompatible” or not is determined by the following compatibility test.

(Compatibility Test)

Each 10 g of two oils is placed in a transparent glass container having a volume of 30 mL (screw tube No. 6 manufactured by Maruemu Corporation) and heated to 90° C. Then the container is covered with a lid, subsequently is shaken vigorously up and down 30 times, and is allowed to stand in a room at 25° C. After 24 hours, a state of the content is visually observed at 25° C. to determine “compatible” or “incompatible”. When the state corresponds to the score of 1 described below, it is determined to be “compatible”, and when the state corresponds to the score of 2 or 3 described below, it is determined to be “incompatible.”

Score 1: The content has a single phase, which is transparent and homogeneous.

Score 2: The content is cloudy entirely or has at least one of an upper layer and a lower layer which is transparent, and a cloudy intermediate layer.

Score 3: The content has an upper layer and a lower layer, both of which are transparent, and a clear boundary between the upper layer and the lower layer or contains clear droplets of visible size.

As the silicone oil of the component (B), dimethicone (dimethylpolysiloxane) being liquid at 25° C. or dimethiconol is preferably used. Specific examples of commercially available silicone oils include dimethicone of KF-96 series manufactured by Shin-Etsu Chemical Co., Ltd. and dimethicone of SH200 series manufactured by Dow Toray Co., Ltd., and the like. Of these, dimethicone is preferred because of its low compatibility with the oil of component (A). A kinematic viscosity at 25° C. of the silicone oil is preferably 6 to 100,000 mm²/s, more preferably 20 to 50,000 mm²/s, and more preferably 100 to 10,000 mm²/s. If the viscosity is too low, the silicone oil tends to be compatible with the oil of the component (A), while if the viscosity is too high, application to the skin or lips tends to become difficult. When the component (B) is dimethiconol, the viscosity at 25° C. is preferably 1,000 mPa·s or more.

A content of the silicone oil of the component (B) in the oil-based cosmetic of the present invention is preferably 5 to 70% by mass, more preferably 5 to 60% by mass, furthermore preferably 10 to 50% by mass. If the silicone oil content is too small, color transfer resistance tends to decrease, and if too large, dispersibility of colorants tends to decrease.

In the oil-based cosmetic of the present invention, a ratio by mass of a blending amount of the component (A) to a blending amount of the component (B), that is, [(A)/(B)] is preferably 1/10 to 1/0.1, more preferably, ⅕ to 1/0.2, furthermore preferably ½ to 1/0.5. When the ratio is too small, gloss persistence tends to decrease, while when too large, color transfer resistance tends to decrease.

The oil-based cosmetic of the present invention preferably contains, as component (C), a separation inhibitor that suppresses separation between the oil of the component (A) and the silicone oil of the component (B) when both components are mixed. By containing this component (C), the oil-based cosmetic tends to form a stable oil-in-oil emulsion layer. When the oil of the component (A) and the silicone oil of the component (B) are homogeneously mixed macroscopically to form a monolayer of an oil-in-oil emulsion, in the case of a liquid cosmetic, the component (A) and the component (B) do not separate. Also, in the case of a solid cosmetic, the solid cosmetic maintains a uniform state until it solidifies after being filled in a molten state. Therefore, it can have a sufficient hardness or a sufficient breaking strength. The separation inhibitor may be any compound as far as it has a function of suppressing separation between the oil of the component (A) and the silicone oil of the component (B) when mixing both components. Whether a compound has a function of suppressing separation of both components or not is determined according to the method shown in Test Example 2 described later, and if a state when 3 hours elapsed after standing a mixture of the component (A), the component (B) and the compound corresponds to the score of 3 in the criteria (i.e., when at least one of an upper layer and a lower layer is a transparent or translucent layer, and cloudy layer is 40% by volume or more and less than 80% by volume of the whole becomes cloudy.) or more, the compound is determined to have a separation suppression function. The separation inhibitor used is preferably one or more selected from the group consisting of the following components (C-1), (C-2), (C-3) and (C-4).

<(C-1) Lipophilic Surfactant Having an HLB Value of 6 or Less>

HLB is an index of a balance between hydrophilicity and lipophilicity at a value of from 0 to 20, indicating that the closer to 0, the higher the lipophilicity, and the higher hydrophilicity, the closer to 20. Various calculation methods are known as a method for calculating an HLB value, and HLB values are sometimes described in catalogs and the like provided by manufacturers. In the present specification, an HLB value of a lipophilic surfactant is the same as the HLB value described in a catalog provided by a manufacturer if it is commercially available, and if not commercially available, its HLB value is determined in accordance with the method of Griffin described on page 307 of “Handbook of surfactants” published from Sangyo Tosho Publishing Co. Ltd. in 1960. The method is represented by the following formula.

HLB value=20×(total molecular weight of hydrophilic portion/total molecular weight)

The lipophilic surfactant having an HLB value of 6 or less of (C-1) is a nonionic surfactant. Specifically, there are exemplified glyceryl fatty acid esters such as glyceryl stearate and glyceryl diisostearate; polyglyceryl fatty acid esters such as polyglyceryl-2 isostearate, polyglyceryl-2 diisostearate, and polyglyceryl-2 oleate; sorbitan fatty acid esters such as sorbitan isostearate, sorbitan sesquiisostearate, sorbitan oleate, and sorbitan sesquioleate: propylene glycol fatty acid esters such as propylene glycol stearate; ethylene glycol fatty acid esters such as glycol stearate; silicone surfactants such as polyether-modified silicone and polyglycerin-modified silicone; and the like. Of these, sorbitan sesquiisostearate and sorbitan sesquioleate are preferably used from the viewpoint of stability of the oil-in-oil emulsion.

<(C-2) Ester of Polyhydric Alcohol and Condensed Hydroxy Fatty Acid>

An ester of polyhydric alcohol and condensed hydroxy fatty acid is an ester obtained by reacting a condensed hydroxy fatty acid with a polyhydric alcohol. Specific examples of the polyhydric alcohol used as a starting material for such an ester include alkane polyols such as ethylene glycol, pentaerythritol and glycerin; sugars such as sucrose; sugar derivatives such as sugar alcohols such as sorbitol and mannitol; polyethylene glycol; polyglycerins such as pentaglycerin, hexaglycerin, decaglycerin; polyalkanepolyols such as dipentaerythritol and tripentaerythritol; and the like. These compounds are used as solely or as a mixture of two or more thereof. Among the polyhydric alcohols, polyglycerin, pentaerythritol and dipentaerythritol are preferred, and polyglycerin having a degree of polymerization of 2 to 10, especially 4 to 8 is more preferred.

A condensed hydroxy fatty acid used as the other starting material of the ester is a condensation product of a hydroxy fatty acid. A degree of condensation of the product is usually greater than 1, preferably three or more. The hydroxy fatty acid is a fatty acid having one or more hydroxyl groups in its molecule. Specific examples thereof include ricinoleic acid, 12-hydroxystearic acid, hydrogenated castor oil fatty acid (fatty acid containing 12-hydroxystearic acid and a small amount of stearic acid and palmitic acid), 12-hydroxydecanoic acid, iprolic acid, 2-hydroxyhexadecanoic acid, 11-hydroxyhexadecanoic acid, 16-hydroxyhexadecenoic acid, 2-hydroxystearic acid, 9-hydroxystearic acid, 10-hydroxystearic acid, 18-hydroxystearic acid, 9,10-dihydroxystearic acid, and the like. They may be used alone or as a mixture of two or more thereof. Preferably, the hydroxy fatty acid has carbon atoms of 8 to 22, more preferably 12 to 20. Among them, ricinoleic acid, 12-hydroxystearic acid, 9-hydroxystearic acid, 10-hydroxystearic acid and hydrogenated castor oil stearic acid are preferred, and ricinoleic acid and 12-hydroxystearic acid are particularly preferred.

Specific examples of the ester include polyglyceryl condensed ricinoleates such as tetraglyceryl condensed ricinoleate (INCI name: Polygriceryl-4 polyricinoleate), pentaglyceryl condensed ricinoleate (INCI name: Polyglyceryl-5 polyricinoleate), hexaglyceryl condensed ricinoleate (INCI name: Polyglyceryl-6 polyricinoleate), decaglyceryl condensed ricinoleate (INCI name: Polyglycery-10 polyricinoleate); polyglyceryl condensed 12-hydroxystearates such as hexaglyceryl condensed 12-hydroxystearate (INCI name: Polyglyceryl-6 polyhydroxystearate), diglyceryl di-condensed 12-hydroxystearate (INCI name: Polyglyceryl-2 dipolyhydroxystearate); dipentaerythrityl tripolyhydroxystearate; pentaerythritol condensed ricinoleate: pentaerythritol condensed 12-hydroxystearate: tripentaerythritol condensed ricinoleate: tripentaerythritol condensed 12-hydroxystearate; polyethylene glycol condensed ricinoleate; polyethylene glycol condensed 12-hydroxystearates such as PEG-30 di-condensed 12-hydroxystearate (INCI name: PEG-30 dipolyhydroxystearate); and the like. They can be used solely or as a mixture of two or more thereof. Of these, esters of condensed ricinoleic acid or condensed 12-hydroxystearic acid and polyglycerin having a degree of polymerization of 2 to 10 or dipentaerythritol are preferably used.

Examples of commercially available products of the ester include SUN SOFT No. 818 R—C(Polyglyceryl-5 polypolyricinoleate, manufactured by Taiyo Kagaku Co., Ltd.), HEXGLYN PR-15 (Polyglyceryl-6 polypolyricinoleate, manufactured by Nikko Chemicals Co. Ltd), DECAPILN PR 20 (polyricinoleic acid polyglyceryl-10, manufactured by Nikko Chemicals Co., Ltd.), and CRESTER PR (Polyglyceryl-3 polyricinoleate, manufactured by CRODA), any of which is a polyglycerol condensed hydroxystearate; DEHYMULS PGPH (dipolyhydroxystearate polyglyceryl-2, manufactured by Cognis Holding) which is a poly glycerol hydroxystearate; SALACOS WO-6 (dipentaerythrityl tri-polyhydroxystearate, manufactured by The Nisshin OilliO Group, Ltd.) which is a dipentaerythritol condensed 12-hydroxystearate; CITHROL DPHS manufactured by CRODA which is a polyethylene glycol condensed 12-hydroxystearate; and the like.

<(C-3) Dipentaerythritol Ester of 12-Hydroxystearic Acid or Mixed Fatty Acid Composed of 12-Hydroxystearic Acid and Fatty Acid Having Carbon Atoms of 12 to 22>

Specific examples of the ester of dipentaerythritol and 12-hydroxystearic acid include dipentaerythritol hexahydroxystearate, dipentaerythritol tetrahydroxystearate, and the like. Specific examples of the ester of dipentaerythritol and a mixed fatty acid of 12-hydroxystearic acid and a fatty acid having carbon atoms of 12 to 22 include dipentaerythrityl tetrahydroxystearate/tetraisosteariate, dipentaerythrityl pentahydroxystearic/pentaisostearate, dipentaerythrityl hexahydroxystearte/hexastearate/hexarosinate, and the like.

Examples of the component (C-3) which are available commercially include COSMOL 168M (dipentaerythrityl tetrahydroxystearate/tetraisostearate), COSMOL 168EV (dipentaerythrityl hexahydroxystearate), COSMOL 168AR-V (dipentaerythrityl hexahydroxyystearate/hexastearate/hexarosinate) any of which is mamufactured by The Nisshin OilliO Group, Ltd. Of these, dipentaerythrityl tetrahydroxystearate/tetraisostearate is preferred in view of stability of the oil-in-oil emulsion.

Dipentaerythritol has six hydroxyl groups in its molecule. It is preferred that some of the hydroxyl groups have not been esterified in view of stability of the oil-in-oil emulsion. Tetraesters and pentaesters of dipentaerythritol are more preferred. Specifically, dipentaerythrityl tetrahydroxystearate, dipentaerythrityl tetrahydroxysteariate/tetraisostearate, dipentaerythrityl pentahydroxystearate/pentaisostearate and the like are preferably used.

<(C-4) Aerosol Silica and/or Organically Modified Clay Mineral>

Aerosol silica is a fine amorphous silica, also referred to as fumed silica, which is a light and white powder having a fluffy appearance. The aerosol silica can be obtained, for example, by subjecting a raw material such as silicon tetrachloride to high temperature hydrolysis in oxyhydrogen flame. A specific surface area of the aerosol silica is preferably 30 m²/g or greater, more preferably 50 to 400 m²/g, furthermore preferably 100 to 400 m²/g. When the specific surface area is too small, contribution of the aerosol silica to stability of the oil-in-oil emulsion tends to decrease.

A primary particle size of the aerosol silica is preferably 50 nm or less, more preferably 20 nm or less. The primary particle size is determined as an average value of 3,000 to 5,000 particles measured by an electron micrograph. The aerosol silica may be an untreated silica exhibiting hydrophilicity, or a hydrophobized silica subjected to a hydrophobization treatment. Specific examples of the hydrophobization treatment include dimethyldichlorosilane treatment, trimethylsiloxy treatment using trimethylsilyl chloride or hexamethyldisilazane, octylsilane treatment, dimethyl silicone oil treatment, coating and baking treatment using methyl hydrogen polysiloxane, coating with metal soap, and the like.

Examples of commercial products of the aerosol silica include, as the untreated aerosol silica, AEROSIL 50, AEROSIL 130, AEROSIL 200, AEROSIL 200V, AEROSIL 200CF, AEROSIL 200FAD, AEROSIL 300, AEROSIL 300CF, AEROSIL 380, AEROSIL 380S, any of which is manufactured by Nippon Aerosil Co., Ltd. and the like, and include, as the hydrophobized aerosol silica, AEROSIL R972, AEROSIL R972V, AEROSIL R972CF, AEROSIL R974, AEROSIL R976S, AEROSIL RX200, AEROSIL RX 300, AEROSIL RY 200, AEROSIL R202, AEROSIL R805, AEROSIL R812, AEROSIL RA200H, any of which is manufactured by Nippon Aerosil Co., Ltd., CAB-O-SIL TS530 which is manufactured by Cabot Corporation, and the like.

Organically modified clay minerals are materials in which cations of smectite that is a swellable layered silicate are substituted with an organic cation such as a quaternary ammonium salt. Examples of minerals that are considered as “smectite” include hectorite, montmorillonite, bentonite, saponite, beidellite, nontronite, stevensite, vermiculite, volkonskoite, sauconite, magadite, kenyalite, and the like. Examples of the organic cation include dimethyldistearyl ammonium chloride, dimethyldipalmityl ammonium chloride, stearyl benzyl dimethyl ammonium chloride, stearyl trimethylammonium chloride, lauryl dimethylaminoacetic acid betaine, and the like. Disteardimonium hectorite (INCI name) in which the cations of hectorite are substituted with dimethyldistearyl ammonium cation is particularly preferably used. An example of disteardimonium hectorite available commercially is BENTONE 38V manufactured by Elementis Specialties Inc.

The component (C) is preferably used in combination of multiple components selected from the group consisting of (C-1) to (C-4) in view of stability of the oil-in-oil emulsion. Preferable combinations include a combination of the component (C-2) and the component (C-3), and a combination of the component (C-2), the component (C-3) and the component (C-4). A blending amount of the component (C) is preferably 0.1 to 40% by mass, more preferably 1 to 30% by mass relative to the whole cosmetic. The incorporation of the component (C) facilitates maintaining uniformity of the cosmetic. The blending amount is preferably 0.2 to 80 parts by mass per 100 parts by mass of the total amount of the oil of the component (A) and the silicone oil of the component (B).

The oil-based cosmetic of the present invention may contain a powder as component (D). If the cosmetic contains the powder, it enables the skin and lips to color, and texture of finish upon application can be adjusted. In the case of a makeup cosmetic containing a coloring powder, it is excellent in gloss, color transfer resistance and color retention. The powder is not particularly limited as far as used usually in conventional cosmetics and can be used regardless of its shape (spherical, needle-like, plate-like), particle size (aerosol, fine particulate, pigment grade, etc.), particle structure (porous, non-porous, etc.). It is noted that, in the present specification, the aerosol silica and the organically modified clay mineral any of which is the component (C-4) are not included in the component (D).

As the coloring powder, inorganic coloring powders and organic coloring powders are used. Examples of the inorganic coloring powder include inorganic white pigments such as titanium oxide, zinc oxide; inorganic red pigment such as iron oxide, iron hydroxide, and iron titanate; inorganic brown pigments such as gamma-iron oxide; inorganic yellow pigments such as iron yellow oxide and ocher; inorganic black pigments such as black iron oxide and carbon black; inorganic purple pigments such as manganese violet and cobalt violet; inorganic green pigments such as chromium hydroxide, chromium oxide, cobalt oxide and cobalt titanate; inorganic blue pigments such as iron blue and ultramarine blue; glittering pigments such as mica titanium, titanium oxide-coated glass flakes and aluminum powder; and the like.

Examples of the organic coloring powders include organic pigments such as Red No. 201, Red No. 202, Red No. 204, Red No. 226, Red No. 228, Orange No. 203, Orange No. 204, Blue No. 404, and Yellow No. 401; lakes of pigments such as Red No. 104, Yellow No. 4, Yellow No. 5, and Blue No. 1; and lakes of natural pigments such as carminic acid, laccaic acid, and carthamin.

An extender powder is used, as appropriate, to adjust feeling in use such as stickiness and dry feeling. Examples of the extender powder include inorganic extender pigments such as talc, white mica, synthetic mica, phlogopite, synthetic fluorine phlogopite, sericite, zeolite, kaolin, bentonite, clay, silicic acid, silicic anhydride (silica), magnesium silicate, aluminum magnesium silicate, calcium silicate, barium sulfate, magnesium carbonate, boron nitride, bismuth oxychloride, alumina, zirconium oxide, and hydroxyapatite; organic extender powders such as silicone powder, silicone elastic powder, polyurethane powder, cellulose powder, nylon powder, silk powder, PMMA powder, starch, polyethylene powder, lauroyl lysine, and metal soap; and composites thereof.

A blending amount of the component (D) to be used in the present invention is preferably 0.01 to 70% by mass, more preferably 0.1 to 50% by mass, furthermore preferably 1 to 30% by mass relative to the whole composition. If the blending amount of the component (D) is too large, color transfer resistance tends to decrease.

The oil-based cosmetic of the present invention may contain a solid oil, a lipophilic gelling agent or both thereof as component (E). Such the component (E) imparts viscosity or hardness to the oil-based cosmetic, so that it contributes to stability of the cosmetic by preventing sedimentation of powders when the cosmetic is liquid, and it also contributes to shape retention in a form such as stick and cake when the cosmetic is solid. The solid oil is solid at room temperature, and its melting point is usually from 50 to 120° C., preferably from 55 to 105° C., more preferably from 60 to 100° C. The melting point of the solid oil can be measured according to the melting point measurement method (second method), which is the general test method defined in Japanese Standards of quasi-drug ingredients. If a solid oil with an excessively low melting point is used, shape retention tends to decrease and if a solid oil with an excessively high melting point is used, operations at an elevated temperature which are disadvantageous in terms of production are required.

The solid oil specifically includes hydrocarbon waxes such as paraffin wax, polyethylene wax, ethylene propylene copolymer, microcrystalline wax, ceresin, ozokerite, synthetic wax, Fischer-Tropsch wax; waxes such as Japan wax, carnauba wax, candelilla wax, rice wax, beeswax; hydrogenated jojoba oil, hardened oil, higher alcohol, synthetic alcohol having an alkyl chain with carbon atoms of 20 to 50, silicone wax, and the like.

Examples of the solid oil which is commercially available include paraffin waxes such as PARAFFIN WAX 135, PARAFFIN WAX 140, PARAFFIN WAX 150 and HNP-11 any of which is manufactured by Nippon Seiro Co., Ltd.; microcrystalline waxes such as HNP-9, HI-MIC-2065, HI-MIC-1070, HI-MIC-1080, HI-MIC-1090 and HNP-0190 (any of which is manufactured by Nippon Seiro Co., Ltd.) and MULTIWAX W-445 which is manufactured by Sonnenborn LLC; polyethylene waxes such as PERFORMALENE 400, PERFORMALENE 500 and PERFORMALENE 655 any of which is manufactured by New Phase Technologies Inc., synthetic waxes (Fischer-Tropsch waxes) such as CIREBELLE 108, CIREBELLE 305 (any of which is manufactured by Cirebelle Inc.) and SASOLWAX TITANEL which is manufactured by Sasol Limited; candelilla waxes such as REFINED CANDELLILLA WAX NO. 1, CANDELLILLA NC 1630 (any of which is manufactured by Cerarica Noda Co., Ltd.), REFINED CANDELLILLA WAX CG-7, REFINED CANDELLILLA WAX SR-3 (any of which is manufactured by Yokozeki Oil & Fat Industries Co., Ltd.), REFINED CANDELLILLA WAX CG-7, REFINED CANDELLILLA WAX SR-3 and HIGH MELTING POINT CANDELLILLA WAX 100 (any of which is manufactured by Japan Natural Products Co., Ltd.); and the like.

The lipophilic gelling agent is a material that can be dissolved or dispersed in a liquid oil at room temperature and can act to thicken or gel the oil at the temperature. Examples of the lipophilic gelling agent include dextrin fatty acid esters, inulin fatty acid esters, sucrose fatty acid esters, starch fatty acid esters, metal soaps, 12-hydroxystearic acid, and the like.

The dextrin fatty acid ester is an ester of dextrin or reduced dextrin and a higher fatty acid. Examples thereof include dextrin palmitate, dextrin palmitate/2-ethylhexanoate, and the like. Examples of commercially available dextrin palmitate include RHEOPEARL KL2 and RHEOPEARL TL2 any of which is manufactured by Chiba Flour Milling Co., Ltd. Examples of commercially available dextrin palmitate/2-ethylhexanoate include RHEOPEARL TT2 which is manufactured by Chiba Flour Milling Co., Ltd. The inulin fatty acid ester is an ester of inulin, which is a kind of fructooligosaccharides, and a higher fatty acid. The inulin preferably has an average molecular weight of 300 to 10,000. Specific examples of the inulin fatty acid ester include inulin stearate, and examples of commercially available products include RHEOPEARL ISK2 which is manufactured by Chiba Flour Milling Co., Ltd. The sucrose fatty acid ester is an oil-soluble sucrose fatty acid ester, and specific examples thereof include sucrose stearate, sucrose tetrastearate triacetate, and the like. Examples of commercially available products include SUGARWAX S-10E, COSMELIKE S-10, SUGARWAX A-10E, any of which is manufactured by DKS Co., Ltd., and the like. Examples of the metal soap include aluminum stearate, calcium stearate, and the like.

The component (E) may be a single compound or two or more compounds in combination as appropriate. A content of the component (E) is usually 1 to 40% by mass, preferably 3 to 30% by mass, more preferably 5 to 25% by mass relative to the total amount of the oil-based cosmetic. If the content of the component (E) is too small, a resultant oil-in-oil emulsion tends to separate or powders tends to settle, resulting in a decrease in stability of the cosmetic, while if too large, a resultant cosmetic tends to be less likely to spread upon application. In the case of preparing a solid oil-based cosmetic, it is preferred to use a solid oil having a melting point of 50 to 120° C. as the component (E), thereby obtaining particularly good shape retention.

The oil-based cosmetic composition of the present invention may contain components used in ordinary cosmetics in addition to the essential components and optional components described above. That is, there may be contained liquid non-volatile oils at room temperature other than the components (A) and (B), volatile oils, oil-soluble resins, surfactants not corresponding to the component (C-1), water, polyhydric alcohols (e.g., glycerin, propylene glycol, butanediol, etc.), lower alcohols (e.g., ethanol, isopropyl alcohol, etc.), UV absorbers (e.g., dimethicodiethylbenzalomalonate, ethylhexyl methoxycinnamate, etc.), moisturizers, fragrances, antioxidants, preservatives, antifoaming agents, fibers, various extracts and the like within a range in which the effects of the present invention are not impaired. Examples of the surfactant other than the component (C-1) include nonionic surfactants having an HLB value of more than 6, anionic surfactants, amphoteric surfactants, and cationic surfactants. Dimethicodiethylbenzalomalonate which is an ultraviolet absorber is preferably used because it contributes to stabilization of the oil-in-oil emulsion.

The oil-based cosmetic of the present invention can be prepared according to conventional methods. For example, all ingredients are heated to a temperature of their melting points or higher, uniformly mixed, poured into a container or a mold in a molten state, and cooled or allowed to stand to obtain the oil-based solid cosmetic. Also, a liquid or pasty cosmetic can be obtained by heating all ingredients to a temperature of their melting points or higher and then cooling a mixture while stirring. Further, the oil-based cosmetic can be obtained by preliminarily kneading coloring materials with a part of oily components and/or surfactant components and then mixing with the rest of the ingredients under heating.

Examples of applications of the oil-based cosmetic of the present invention, when containing a coloring powder, include a lipstick, a lip gloss, an eye shadow, a face color, a concealer, a foundation, a makeup base, a sunscreen, a hair colorant, and the like. Among them, the oil-based cosmetic is particularly suitable for a lipstick and a lip gloss. In addition, when the oil-based cosmetic of the present invention contains no coloring powder, it can be used as a topcoat for a makeup formed by a makeup cosmetic containing a coloring powder. In that case, gloss, color transfer resistance, and cosmetic retention of the makeup cosmetic containing a coloring powder are improved due to presence of the topcoat layer. Examples of a makeup cosmetic which is suitable for using the topcoat include a lipstick, a foundation, an eye color, and the like. The topcoat is particularly suitable as a lipstick topcoat that is also referred to as a lipstick overcoat or a lip coat. A form of the oil-based cosmetic of the present invention can be appropriately selected according to its application. For example, it may be liquid, pasty, solid, or the like. Oil-based solid cosmetics that are used by being filled in a metal plate, a resin plate, a stick-like container, etc. have advantages of being easy to use and easy to carry.

### EXAMPLE

Hereinafter, the present invention will be further described with reference to Examples, but the present invention is not limited by these Examples. Test Example 1 described below illustrates the method for determining whether an oil corresponds to the oil of the component (B) used in the present invention or not, while Test Example 2 illustrates the method for determining whether a compound corresponds to the separation inhibitor of the component (C) which is an optional component or not. “Parts” and “%” in Examples, Comparative Examples and Test Examples represent “parts by mass” and “% by mass” relative to the total amount unless otherwise specified. Evaluation method for the oil-based cosmetics in the following Examples and Comparative Examples is as follows.

(Evaluation Item and Evaluation Method)

I Color Transfer Resistance:

Ten evaluators applied a sample to their lips, and 10 minutes later, they used a coffee cup. Color transfer resistance was evaluated based on a color strength of a lip mark formed on the cup.

II Color Retention:

Ten evaluators applied a sample to their lips and evaluated sensorily in 5 stages based on the following scoring standard (score).

III Gloss Immediately after Application:

Ten evaluators applied a sample to their lips and evaluated sensorily in 5 stages based on the following scoring standard (score).

IV Gloss Persistence:

Ten evaluators applied a sample to their lips and evaluated sensorily in 5 stages based on the following scoring standard (score).

V Moldability Upon Filling:

A solid sample was subjected to a breaking strength test to evaluate moldability upon filling. That is, a sample filled and molded in a stick container with a diameter of 10 mm was held horizontally on a specimen mount of a rheometer (FUDOH RHEOMETER manufactured by K.K. Rheotech) in an environment of 30° C., and a T-shaped plunger was applied to the position that is 10 mm apart from the tip of the middle plate of the stick, while the specimen mount was raised at a speed of 2 cm/min. A stress (gf) at which the stick broke was measured and taken as breaking strength. When the sample is solidified in a uniform state, the breaking strength value becomes high, and when the sample causes separation of the oil-in-oil emulsion before solidification, the breaking strength value becomes low. When the breaking strength value (gf) was 80 or more, moldability upon filling was determined as S; when the breaking strength value (gf) was 50 or more and less than 80, it was determined as A; when the breaking strength value (gf) was 30 or more and less than 50, it was determined as B; and when the breaking strength value (gf) was less than 30, it was determined as C.

VI Separation Stability (Stability Against Separation)

A liquid or semi-solid sample was placed in a glass bottle and stored in a constant temperature bath at 50° C. When separation was not observed for a period of 2 weeks or more, separation stability was determined as S, when separation was not observed for a period of 1 week or more and less than 2 weeks, it was determined as A, when separation was not observed for a period of 2 days or more and less than 1 week, it was determined as B, and when separation was observed within one day, it was determined as C.

(Score)

5: Very good

4: Good

3: Ordinary

2: Poor

1: Very poor

(Evaluation Criteria)

S: Average value of scores by ten evaluators is 4.0 or more.

A: Average value of scores by ten evaluators is 3.5 or more and less than 4.0.

B: Average value of scores by ten evaluators is 2.5 or more and less than 3.5.

C: Average value of scores by ten evaluators is less than 2.5.

### Test Example 1

(Compatibility Test)

Compatibility of Sample 1 described below that corresponds to the oil of the component (A) and each of liquid oils commonly used in conventional cosmetics (Samples 2-1 to 2-5 and 3-1 to 3-6 below) was evaluated according to the following test method and criteria. The results are shown in Table 1. As the oil to be evaluated, liquid oils commonly used in cosmetics were selected.

Test Method

After 10 g of the oil of the following Sample 1 and 10 g of the oil to be evaluated were placed in a transparent glass container having a volume of 30 mL (screw tube No. 6 manufactured by Maruemu Corporaion), the content of the container was heated to 90° C. Then the container was covered with a lid, subsequently was shaken vigorously up and down 20 times, and was allowed to stand in a room at 25° C. After 24 hours, a state of the content was visually observed at 25° C. to rank based on the following criteria. When a state of the content corresponds to the score of 1, both compounds are determined to be “compatible”, and when a state of the content corresponds to the score of 2 or 3, both compounds are determined to be “incompatible.”

Criteria

Score 1: The content has a single phase which is transparent and homogeneous.

Score 2: The content is cloudy entirely or has at least one of an upper layer and a lower layer which is transparent, and a cloudy intermediate layer.

Score 3: The content has an upper layer and a lower layer both of which are transparent and a clear boundary between the upper layer and the lower layer or contains clear droplets of visible size.

The samples provide to the test are as follows.

Sample 1 [Oil of the component (A)]:

A liquid oil at 25° C. having a steradiene content of 90% or more and a refractive index at 40° C. of 1.52 that is prepared according to the description of Example 2 in Japanese Patent No. 6,353,939 (Patentee: Yokozeki Oil & Fat Industries Co., Ltd) by subjecting a phytosterol obtained from soybean oil and rapeseed oil to a catalytic dehydration reaction in the presence of p-toluenesulfonic acid monohydrate to remove the hydroxyl group at 3-C of the phytosterol.

Sample 2-1:
Dimethicone having a kinematic viscosity at 25° C. of 20 mm²/s (trade name: KF-96A-20cs, manufactured by Shin-Etsu Chemical Co. Ltd.)

Sample 2-2:
Dimethicone having a kinematic viscosity at 25° C. of 100 mm²/s (trade name: KF-96A-100cs, manufactured by Shin-Etsu Chemical Co. Ltd.)

Sample 2-3:
Dimethicone having a kinematic viscosity at 25° C. of 1,000 mm²/s (trade name: KF-96-1000cs, manufactured by Shin-Etsu Chemical Co. Ltd.)

Sample 2-4:
Dimethicone having a kinematic viscosity at 25° C. of 3,000 mm²/s (trade name: KF-96-3000cs, manufactured by Shin-Etsu Chemical Co. Ltd.)

Sample 3-1:
Dimethicone having a kinematic viscosity at 25° C. of 2 mm²/s (trade name; KF-96L-2cs, manufactured by Shin-Etsu Chemical Co. Ltd.)

Sample 3-2:
Diphenylsiloxyphenyl trimethicone having a kinematic viscosity at 25° C. of 15 mm²/s (trade name: KF-56A, manufactured by Shin-Etsu Chemical Co. Ltd.)

Sample 3-3:
Diphenyldimethicone having a kinematic viscosity at 25° C. of 400 mm²/s (trade name: KF-54, manufactured by Shin-Etsu Chemical Co. Ltd.)

Sample 3-4:
Isononyl isononanoate that is an ester oil (trade name: SALACOS 99, manufactured by The Nissin OilliO Group Co., Ltd.)

Sample 3-5:
Triethylhexoin that is an ester oil (trade name: T.I.O, manufactured by The Nissin OilliO Group Co., Ltd.)

Sample 3-6:
Polyisobutene that is a hydrocarbon oil (trade name: PARLREAM24, manufactured by NOF Corporation)

The results of the compatibility test (compatibility with the oil of Sample 1) are shown in Table 1 below.

TABLE 1

| | Sample | Scor on Copatibility |
|---|---|---|
| 2-1 | Dimethicone 20 mm²/s | 3 (Incompatible) |
| 2-2 | Dimethicone 100 mm²/s | 3 (Incompatible) |
| 2-3 | Dimethicone 1000 mm²/s | 3 (Incompatible) |
| 2-4 | Dimethicone 3000 mm²/s | 3 (Incompatible) |
| 3-1 | Dimethicone 2 mm²/s | 1 (Compatible) |
| 3-2 | Diphenylsiloxyphenyl trimethicone | 1 (Compatible) |
| 3-3 | Diphenyldimethicone 400 mm²/s | 1 (Compatible) |
| 3-4 | Isononyl isononanoate | 1 (Compatible) |
| 3-5 | Triethylhexoin | 1 (Compatible) |
| 3-6 | Polyisobutene | 1 (Compatible) |

From the results shown in Table 1, it was confirmed that silicone oils have different compatibility with the oil of Sample 1 depending on their viscosity, and that a phenyl-modified silicone, an ester oil, and a hydrocarbon oil are compatible with the oil of Sample 1. Also, when a commercially available oil (trade name: TECHNOL SD with a refractive index of 1.52 that is manufactured by Yokozeki Oil & Fat Industries Co., Ltd. and has an INCI name of Phytosterols.) was used instead of Sample 1 as the oil of the component (A), similar results to those of the test using Sample 1 as the oil of the component (A) were obtained.

### Test Example 2

<Test for Stabilization of Oil-In-Oil Emulsion (Separation Suppression Effect)>

A third component was added to a mixture of the oil of the component (A) and the silicone oil of the component (B) which are incompatible. Whether or not the third component has an effect of suppressing separation between the oil of the component (A) and the silicone oil of the component (B) was evaluated according to the following test method and criteria. The Results are shown in Table 2.

Test Method

After 10 g of the oil of the component (A) (Sample 1 described above) and 10 g of the silicone oil of the component (B) that is dimethicone having a kinetic viscosity at 25° C. of 1,000 mm²/s (trade name: KF-96-100, manufactured by Shin-Etsu Chemical Co. Ltd.) were placed in a transparent glass container having a volume of 30 mL (screw tube No. 6 manufactured by Maruemu Corporaion), 4 g of a compound to be evaluated (i.e., one of Samples 4-1 to 4-6 and 5-1 to 5-3 described below) was added to the mixture, and heated to 90° C. Then the container was covered with a lid, subsequently was shaken vigorously up and down 20 times, and was allowed to stand in a room at 25° C. After 3 hours and 24 hours, a state of the content was visually observed at 25° C.

Criteria

Uniformly cloudy.

4: At least one of an upper layer and a lower layer is a transparent or translucent layer, and 80% by volume or more of the whole becomes cloudy.

3: At least one of an upper layer and a lower layer is a transparent or translucent layer, and 40% by volume or more and less than 80% by volume of the whole becomes cloudy.

2: At least one of an upper layer and a lower layer is a transparent or translucent layer, and 1% by volume or more and less than 40% by volume of the whole becomes cloudy.

1: Both upper and lower layers are transparent and has a clear boundary in the middle, or transparent droplets of visible size are observed.

In the present invention, when a state of the content after 3 hours elapsed after standing corresponds to the score of 3 or more (i.e., 3, 4, or 5) in the above criteria, it is determined that “the compound to be evaluated has a separation suppression function.”

The samples provided to the test are as follows.

Sample 4-1:
Sorbitan sesquiisostearate (trade name: SI-15R, manufactured by Nikko Chemical Co. Ltd.)

Sample 4-2:
Polyglyceryl-2 diisostearate (trade name: COSMOL 42V, manufactured by The Nissin OilliO Group Co., Ltd.)

Sample 4-3:
Polyglyceryl-5 polyricinoleate (trade name: SUNSOFT No. 818R—C, manufactured by Taiyo Kagaku Co. Ltd.)

Sample 4-4:
Dipentaerythrityl tripolyhydroxystearate (trade name: SALACOS WO-6, manufactured by The Nissin OilliO Group Co., Ltd.)

Sample 4-5:
Dipentaerythrityl tetrahydroxystearate/tetraisostearate (trade name: COSMOL 168EV, manufactured by The Nissin OilliO Group Co., Ltd.)

Sample 4-6:
Aerosol silica (trade name: AEROSIL 300, manufactured by Nippon Aerosil Co., Ltd)

Sample 5-1:
Polyisobutene (trade name: PARLREAM24, manufactured by NOF Corporation)

ample 5-2
Neopentyl glycol dicaprate (trade name: ESTEMOL N-01, manufactured by The Nissin OilliO Group Co., Ltd.)

Sample 5-3:
Diphenylsiloxy phenyl trimethicone (trade name: KF-56A, manufactured by Shin-Etsu Chemical Co. Ltd.)

The results of the test for stabilization of oil-in-oil emulsion (i.e., separation suppression effect by a third component) are shown in Table 2 below.

TABLE 2

| | | Separation Suppression Effect | |
|---|---|---|---|
| Sample | Compound Added | After 3 hours | After 24 hours |
| | None | 1 | 1 |
| 4-1 | Sorbitan sesquiisostearate | 3 | 2 |
| 4-2 | Polyglyceryl-2 diisostearate | 3 | 2 |
| 4-3 | Polyglyceryl-5 polyricinoleate | 3 | 2 |
| 4-4 | Dipentaerythrityl | 3 | 2 |
| | tripolyhydroxystearate | | |
| 4-5 | Dipentaerythrityl | 5 | 5 |
| | tetrahydroxystearate/tetraisostearate | | |
| 4-6 | Aerosol silica | 5 | 4 |
| 5-1 | Polyisobutene | 2 | 1 |
| 5-2 | Neopentyl glycol dicaprate | 1 | 1 |
| 5-3 | Diphenylsiloxy phenyl trimethicone | 1 | 1 |

As shown in Table 2, when each of the compounds of (C-1) to (C-4) that are Samples 4-1 to 4-6 was added to a mixture of the oil being the component (A) and the silicone oil being the component (B) which are incompatible, separation of the component (A) and the component (B) was suppressed. That is, these compounds had a function of suppressing the separation of the oil of the component (A) and the silicone oil of the component (B). In contrast, when no third component was added (none), when polybutene which is a hydrocarbon oil (Sample 5-1) was added, when neopentyl glycol dicaprate which is known as a compatibility modifier between a polybutene and a silicone oil (see Japanese Patent No. 6,184,454) (Sample 5-2) was added, and when diphenylsiloxyphenyl trimethicone which is a kind of silicone oils (Sample 5-3) was added, no separation suppression effect was observed. Also, when a commercially available oil (TECHNOL SD manufactured by Yokozeki Oil & Fat Industries Co., Ltd.) instead of Sample 1 was used as the oil of the component (A), almost the same results as those in the case of Sample 1 were obtained.

### Example 1 and Comparative Examples 1 to 2

(Lipstick)

A stick-shaped lipstick having the formulation shown in Table 3 was prepared according to the following production procedure, and evaluated for color transfer resistance, color retention, gloss, gloss persistence, and moldability upon filling according to the method described above. The results are shown in Table 3.

(Production Procedure)

(1) The ingredients 1 to 14 shown in Table 3 were heated to about 90° C. and uniformly mixed.

(2) The mixture prepared in the above (1) was filled at 80° C. into a stick-shaped container in a molten state and allowed to stand to obtain a solid lipstick.

TABLE 3

| | | | | Comparative | Comparative |
|---|---|---|---|---|---|
| | Component | | Example 1 | Example 1 | Example 2 |
| 1 | A | Oil of the Component (A) (※1) | 25.00 | | 25.00 |
| 2 | | Polyisobutene (※2) | | 25.00 | |
| | B | Dimethicone (1000 mm²/s) (※3) | 25.00 | 25.00 | |
| 4 | | Diphenylsiloxy phenyl trimethicon (※4) | | | 25.00 |
| 5 | E | Synthetic wax (※5) | 3.00 | 3.00 | 3.00 |
| 6 | E | Ceresin | 2.00 | 2.00 | 2.00 |
| 7 | E | Microcrystalline wax (※6) | 2.00 | 2.00 | 2.00 |
| 8 | | Isononyl isononanoate | 18.00 | 18.00 | 18.00 |
| 9 | C-2 | Polyglyceryl-5 polyricinoleate (※7) | 10.00 | 10.00 | 10.00 |
| 10 | C-3 | Dipentaerythrityl tetrahydroxystearate (※8) | 10.00 | 10.00 | 10.00 |
| 11 | D | Red No. 202 | 1.00 | 1.00 | 1.00 |
| 12 | D | Aluminum lake of Yellow No.4 | 2.00 | 2.00 | 2.00 |
| 13 | D | Aluminum lake ofRed No. 104 | 1.00 | 1.00 | 1.00 |
| 14 | C-4 | Aerosol silica (※9) | 1.00 | 1.00 | 1.00 |
| Evaluation | | Color transfer resistance | S | S | C |
| | | Color retention | S | A | C |
| | | Gloss | S | A | B |
| | | Gloss persistence | A | C | C |
| | | Moldability upon filling (breaking strength) | S | A | S |

※1 Trade name TECHNOL SD (Yokozeki Oil & Fat Industries Co., Ltd.)
※2 Trade name PARLREAM24 (NOF Corporation)
※3 Trade name SH200 1000cs (Dow Toray Co., Ltd. )
※4 Trade name KF-56A(Sin-Etsu Cemical Co., Ltd.)
※5 Trade name SASOLWAX TITANEL (SASOL Limited.)
※6 Trade name MULTI WAX (Sonnenborn LLC)
※7 Trade name SUNSOFT No. 818-R-C (Taiyo Kagaku Co. Ltd.)
※8 Trade name COSMOL 168EV(The Nissin OilliO Group Co., Ltd.)
※9 Trade name AEROSIL300 (Nippon Aerosil Co., Ltd.)}

As shown in Table 3, the lipstick of Example 1 containing the oil of the component (A) and the silicone oil of the component (B) that is incompatible therewith had excellent color transfer resistance, color retention, gloss, gloss persistence and moldability upon filling. In contrast, the lipstick of Comparative Example 1, in which the oil of the component (A) was replaced with polyisobutene described in Patent Document 1, was significantly inferior in gloss persistence. In addition, the lipstick of Comparative Example 2 using diphenylsiloxyphenyl trimethicone described in Patent Document 1 as a silicone oil was inferior in color transfer resistance, and inferior in color retention and gloss persistence.

### Examples 2 to 7

(Lipstick)

A solid lipstick having the formulation shown in Table 4 was prepared according to the production procedure described above, and evaluated for color transfer resistance, color retention, gloss, gloss persistence and moldability upon filling according to the method described above. The results are shown in Table 4.

TABLE 4

| | | | Example | | | | | |
|---|---|---|---|---|---|---|---|---|
| | Component | Ingredient | 2 | 3 | 4 | 5 | 6 | 7 |
| 1 | A | Oil of the Component (A)(※1) | 25.00 | 25.00 | 25.00 | 25.00 | 25.00 | 25.00 |
| 2 | B | Dimethicone(1000 mm²/s)(※3) | 25.00 | 25.00 | 25.00 | 25.00 | 25.00 | 25.00 |
| 3 | E | Synthetic wax(※5) | 3.00 | 3.00 | 3.00 | 3.00 | 3.00 | 3.00 |
| 4 | E | Ceresin | 2.00 | 2.00 | 2.00 | 2.00 | 2.00 | 2.00 |
| 5 | E | Maicrocrystallin wax(※6) | 2.00 | 2.00 | 2.00 | 2.00 | 2.00 | 2.00 |
| 6 | | Isononyl isononanoate | 18.00 | 18.00 | 18.00 | 18.00 | 19.00 | 39.00 |
| 7 | C-2 | Polyglyceryl-5 polyricinoleate(※7) | 20.00 | | | | 10.00 | |
| 8 | C-3 | Dipentaerythrityl tetrahydroxystearate(※8) | | 20.00 | | | 10.00 | |
| 9 | C-2 | Dipentaerythrityl tripolyhydroxystearate(※10) | | | 20.00 | | | |
| 10 | C-1 | Sorbitan sesquiisostearate(※11) | | | | 20.00 | | |
| 11 | D | Red No. 202 | 1.00 | 1.00 | 1.00 | 1.00 | 1.00 | 1.00 |
| 12 | D | Aluminum lake of Yellow No. 4 | 2.00 | 2.00 | 2.00 | 2.00 | 2.00 | 2.00 |
| 13 | D | Aluminum lake of Red No. 104 | 1.00 | 1.00 | 1.00 | 1.00 | 1.00 | 1.00 |
| 14 | C-4 | Aerosol silica(※9) | 1.00 | 1.00 | 1.00 | 1.00 | | |
| Evaluation | | Color transfer resistance | S | S | S | S | S | S |
| | | Color retention | S | S | S | S | S | S |
| | | Gloss | S | S | S | S | S | S |
| | | Gloss persistence | A | A | A | A | A | A |
| | | Moldability upon filling (breaking strength) | A | A | A | B | A | C |

(※10)Trade name SALACOS WO-6 (The Nissin OilliO Group Co,. Ltd.)
(※11)Trade name SI-15RV (Nikko Chemical Co., Ltd.)

As shown in Table 4, the lipsticks of Examples 2 to 7, which contain the oil of the component (A) and the silicone oil of the component (B) that is incompatible therewith, had excellent color transfer resistance, color retention, gloss, gloss persistence and moldability upon filling. In particular, the lipsticks of Examples 2 to 6 containing a compound corresponding to the components (C-1), (C-2) or (C-3) as the separation inhibitor were superior in the moldability upon filling to the lipstick of Example 7 containing no separation inhibitor. Comparing the lipstick of Example 1 shown in Table 3 and the lipsticks of Examples 2, 3 and 6, the lipstick of Example 1, which contains the components (C-2), (C-3) and (C-4) together as the component (C), was the best in moldability upon filling.

### Example 8 and Comparative Example 3

(Lip Gloss)

A pasty lip gloss having the formulation shown in Table 5 was prepared according to the following production procedure, and evaluated for color transfer resistance, color retention, gloss, gloss persistence, and separation stability according to the method described above. The results are shown in Table 5.

(Production Procedure)

(1) The ingredients 1 to 13 shown in Table 5 were heated to about 90° C. and uniformly mixed.

(2) The mixture prepared in the above (1) was filled into a lip gloss container having a cap fitted with an applicator at 70° C. and allowed to stand to obtain a pasty lip gloss.

TABLE 5

| | | | | Comparative |
|---|---|---|---|---|
| | Component | Ingredient | Example 8 | Example 3 |
| 1 | A | Oil of the component (A) (※1) | 35.00 | 35.00 |
| 2 | B | Dimethicone (2000 mm²/s) (※12) | 20.00 | |
| 3 | | Dimethicone(2 mm²/s) (※14) | | 20.00 |
| 4 | E | Dextrin palmitate/ethylhexanoate (※13) | 5.00 | 5.00 |
| 5 | | Diisostearyl Malate | 16.50 | 16.50 |
| 6 | C-2 | Polyglyceryl-5 polyricinoleate (※7) | 5.00 | 5.00 |
| 7 | C-3 | Dipentaerythrityl tetrahydroxystearate (※8) | 5.00 | 5.00 |
| 8 | D | Red No. 202 | 0.50 | 0.50 |
| 9 | D | Aluminum lake of Yellow No.4 | 0.30 | 0.30 |
| 10 | D | Aluminum lake of Blue No.1 | 0.20 | 0.20 |
| 11 | D | Borosilicate(Ca/Al) coated with TiO₂ and SiO₂) (※14) | 0.50 | 0.50 |
| 12 | D | Borosilicate(Ca/Al) coated with TiO₂ (※15) | 0.50 | 0.50 |
| 13 | C-4 | Aerosol silica | 1.00 | 1.00 |
| Evaluation | Color transfer resistance | | S | C |
| | Color retention | | S | B |
| | Gloss | | S | A |
| | Gloss persistence | | A | C |
| | Separation stability | | S | S |

※12 Trade name SH200 2000cSt (Dow Toray Co., Ltd.)
※13 Trade name RHEOPEARL TT2 (Chiba Flour Milling Co., Ltd.)
※14 Trade name METASHINE MT1080RSS1 (Nippon Sheet Glass Company, Ltd.)
※15 Trade name METASHINE MT1080RGS1 (Nippon Sheet Glass Company, Ltd.)

As shown in Table 5, the lip gloss of Example 8, which contain the oil of the component (A) and the silicone oil of the component, had excellent color transfer resistance, color retention, gloss, gloss persistence and separation stability. On the other hand, the lip gloss of Comparative Example 3, in which the component (B) was replaced with dimethicone having a kinematic viscosity of 2 mm²/s that is compatible with the component (A), had insufficient color transfer resistance and gloss persistence, and further color retention was not satisfactory.

### Example 9

(Double Layer Liquid Lipstick)

A double layer liquid lipstick having the formulation shown in Table 6 was prepared according to the following production procedure, and evaluated for color transfer resistance, color retention, gloss, and gloss persistence according to the method described above. This liquid lipstick separated into two layers within several hours after standing. The upper layer was a layer mainly composed of the oil of the component (A), and the lower layer was an oil-in-oil emulsion layer composed of the oil of the component (A) and the silicone oil of the component (B). In the emulsion layer, separation of the oil of the component (A) and the silicone oil of the component (B) was suppressed by addition of aerosol silica of the component (C-4). The results are shown in Table 6. Incidentally the double layer liquid lipstick is used after homogenizing the content by shaking.

(Production Procedure)

(1) The ingredients 1 to 8 shown in Table 6 were heated to about and uniformly mixed.

(2) The mixture prepared in the above (1) was filled into a lip gloss container having a cap fitted with an applicator and allowed to stand to obtain a double layer liquid lipstick.

TABLE 6

| | Component | Ingredient | Example 8 |
|---|---|---|---|
| 1 | A | Oil of the component (A) (※1) | 35.00 |
| 2 | B | Dimethicone (100 mm²/s) (※16) | 20.00 |
| 3 | | Diisostearyl Malate | 16.50 |
| 4 | | Isononyl isononanoate | 25.00 |
| 5 | D | Red No. 202 | 2.00 |
| 6 | D | Aluminum lake of Yellow No.4 | 0.30 |
| 7 | D | Aluminum lake of Blue No.1 | 0.20 |
| 8 | C-4 | Aeroso silica | 1.00 |
| Evaluation | | Color transfer resistance | S |
| | | Color retention | S |
| | | Gloss | S |
| | | Gloss persistence | A |

※16 Trade name SH200 100cSt (Dow Toray Co., Ltd.)

As shown in Table 6, the liquid lipstick of Example 9, which contains the oil of the component (A) and the silicone oil of the component that is incompatible therewith, had excellent color transfer resistance, color retention, gloss, and gloss persistence.

### Example 10

(Lipstick Topcoat)

A pasty lipstick topcoat having the formulation shown in Table 7 was prepared according to the following production procedure and was applied on lips coated with commercially available lipstick (THREE DARINGLY DEMURE LIPSTICK 02, manufactured by K.K. ARCO). Color transfer resistance, color retention, gloss, gloss persistence, and separation stability were evaluated according to the method described above. The evaluation results are as shown in Table 7.

(Production Procedure)

(1) The ingredients 1 to 7 shown in Table 7 were heated to about and uniformly mixed.

(2) The mixture prepared in the above (1) was filled at 70° C. into a container having a cap fitted with an applicator and allowed to stand to obtain a pasty lipstick topcoat.

TABLE 7

| | Component | Ingredient | Example 10 |
|---|---|---|---|
| 1 | A | Oil of the component (A) (※1) | 35.00 |
| 2 | B | Dimethicone (2000 mm²/s) (※12) | 30.00 |
| 3 | E | Dextrin palmitate/ | 5.00 |
| | | ethylhexanoate (※13) | |
| 4 | | Diisostearyl Malate | 19.00 |
| 5 | C-2 | Polyglyceryl-5 | 5.00 |
| | | polyricinoleate (※7) | |
| 6 | C-3 | Dipentaerythrityl | 5.00 |
| | | tetrahydroxystearate (※8) | |
| 7 | C-4 | Aerosol silica | 1.00 |
| Evalu- | | Color transfer resistance | S |
| ation | | Color retention | S |
| | | Gloss | S |
| | | Gloss persistence | S |
| | | Separation stability | S |

As shown in Table 7, when the lipstick topcoat of Example 10 containing the oil of the component (A) and the silicone oil of the component (B) that is incompatible therewith was applied on a makeup of a lipstick, the makeup exhibited excellent color transfer resistance, color retention, gloss, and gloss persistence. Further, the lipstick topcoat was excellent in separation stability.

### INDUSTRIAL APPLICABILITY

According to the present invention, there is provided an oil-based cosmetic that is excellent in gloss and gloss persistence when applied to the skin or lips in addition to color transfer resistance and color retention.

1. An oil-based cosmetic comprising:
an oil containing a phytosterol derivative which has a refractive index of 1.50 or more at 40° C. and is a liquid state at 25° C. (A); and
a liquid silicone oil being incompatible with the oil (A) at 25° C. (B) as an oily base.

2. The oil-based cosmetic according to claim 1, further comprising a separation inhibitor (C) to suppress separation of the oil (A) and the silicone oil (B).

3. The oil-based cosmetic according to claim 1, further comprising a powder (D).

4. The oil-based cosmetic according to claim 1, further comprising a solid oil and/or a lipophilic gelling agent (E).

5. The oil-based cosmetic according to claim 1, wherein it is an oil-in-oil emulsion cosmetic.

6. The oil-based cosmetic according to claim 1, wherein it contains 5 to 70% by mass of the oil (A) and 5 to 70% by mass of the silicone oil (B), and further a ratio by mass of the component (A) to the component (B)[(A)/(B)] is 1/10 to 1/0.1.

7. The oil-based cosmetic according to claim 1, wherein a content of the separation inhibitor (C) is 0.1 to 40% by mass and is 0.2 to 80 parts by mass per 100 parts by mass of the total amount of the component (A) and the component (B).

8. The oil-based cosmetic according to claim 1, wherein the separation inhibitor (C) is one or more selected from the group consisting of:
(C-1) Lipophilic surfactant having an HLB value of 6 or less;
(C-2) Ester of polyhydric alcohol and condensed hydroxy fatty acid;
(C-3) Dipentaerythritol ester of 12-hydroxystearic acid and/or dipentaerythritol ester of a mixed fatty acid of 12-hydroxystearic acid and a fatty acid having carbon atoms of 12 to 22; and
(C-4) Aerosol silica and/or organically modified clay mineral.
